(19) United States
(12) Patent Application Publication
(10) Pub. No.: US 2019/0076460 A1
(43) Pub. Date: Mar. 14, 2019

(54) An mRNA cancer vaccine encoding human GM-CSF fused to multiple tandem epitopes

(71) Applicant: Enyu Ding, Durham, NC (US)

(72) Inventor: Enyu Ding, Durham, NC (US)

(73) Assignee: Enyu Ding, Durham, NC (US)

(21) Appl. No.: 16/082,718

(22) PCT Filed: Feb. 22, 2017
(86) PCT No.: PCT/US17/18771

§ 371 Date: Sep. 6, 2018

(51) Int. Cl. A61K 31/7105; A61P 35/00; A61K 39/00; C12N 15/113; C12N 15/62; A61K 48/00

(52) U.S. Cl. CPC A61K 31/7105; A61P 35/00; A61K 39/001118; A61K 39/001119; A61K 39/001157; A61K 48/0016; A61K 39/001164; C12N 15/1135; C12N 15/62; A61K 48/0083; A61K 39/001104

(57) ABSTRACT

The present invention provides an mRNA cancer vaccine encoding human GM-CSF fused to multiple tandem epitopes. pVec-GM-CSF-hTes encoding human GM-CSF fused to three tandem hTERT epitopes, pVec-GMKE encoding human GM-CSF fused to three tandem epitopes respectively from MUC1, Kras and EGFR, pVec-hIL-12 encoding human interleukin-12 are respectively constructed, and used as templates for generating the corresponding in vitro transcribed mRNAs, which are mixed together as an mRNA cancer vaccine. This mRNA cancer vaccine contains human GM-CSF used as an immune adjuvant, multiple tandem epitopes constituting as multi-epitope cancer antigens and hIL-12 used to enhance the immunotherapeutic effects.

### BACKGROUND OF THE INVENTION

The present invention in the field of biotechnology relates to a class of mRNA vaccine. In particular, the present invention relates to an mRNA cancer vaccine encoding human granulocyte macrophage colony-stimulating factor (GM-CSF) fused to multiple tandem epitopes.

Therapeutic cancer vaccines that work by stimulating the immune system to fight existing cancers are the most effective drugs to cure cancer because cancer vaccines can elicit the body's immune response and generate immune memory. The first step in ensuring success of cancer vaccines is the antigen design of cancer vaccines. Most antigens used for cancer vaccines are tumor-associated antigens (TAAs), such as human telomerase reverse transcriptase (hTERT), Mucin 1 (MUC1), Kras and epidermal growth factor receptor (EGFR), etc.

A telomere is located at the end of eukaryotic chromosome and is a special “cap” structure composed of tandem repeat non-transcribed DNA sequences (TTAGGG) and telomere-binding proteins. The role of a telomere is to maintain chromosome integrity and control cell division cycle. The telomere of a chromosome becomes shorter with each successive cell division. When a telomere shrinks to a certain extent, the cell stops dividing and is in a quiescent state. Telomerase is an enzyme that can add TTAGGG repeats to the end of chromosomes. Human telomerase catalytic subunit is human telomerase reverse transcriptase (hTERT), which activity is inhibited in normal cells and is too low to be detected. However, in germ cells and stem cells, and especially in the majority of tumor cells (>85%), hTERT is activated and can be abundantly expressed. Therefore, hTERT is the ideal target for cancer treatment.

phTERT DNA vaccine encoding hTERT with two mutated sites is constructed using pGX0001, demonstrating that phTERT DNA vaccine electroporated into the body can break immune tolerance and induce various strong cytotoxic responses in animals [Yan J, et al. Cancer Immunol Res. 2013; 1(3): 179-89]. pGEM4Z/hTERT/A64 and pGEM4Z/hTERT/LAMP/A64 are constructed and used as templates for generating the corresponding in vitro transcribed mRNAs, which are respectively electroporated into dendritic cells (DC). DC-mRNA vaccines are intradermally vaccinated into patients with metastatic prostate cancer. The results show that the chimeric LAMP-hTERT vaccine can elicit significantly higher frequencies of hTERT-specific CD4+ T cells than that with the unmodified hTERT vaccine [Su Z, et al. J Immunol. 2005; 174(6):3798-807]. An adenovirus vaccine encoding hTERT gene (Ad-hTERT) can elicit a strong CD8+ cytotoxic T lymphocyte (CTL) response targeting autologous tumor cells, but adenoviral vectors used for human body may cause significant side effects. In addition, the entire TAA can elicit a strong anti-cancer immune response, but may induce immune tolerance or autoimmune response.

hTERT I540-548 (ILAKFLHWL) is the first hTERT epitope vaccine for melanoma immunotherapy and has entered the phase III clinical trials [Liu J P, et al. Biochim Biophys Acta. 2010; 1805(1): 35-42]. hTERT peptide vaccine GV1001 which is composed of the 16-amino acid residue 611-626 fragment of the hTERT protein can elicit extensive anti-hTERT CD4+ T cell responses in cancer patients [Inderberg-Suso E M, et al. Oncoimmunology 2012; 1(5): 670-686]. A synthetic vaccine comprising hTERT540-548, hTERT572Y-580 and hTERT865-873 tetramer multiple antigen peptides (MAP) is vaccinated into animals, and can elicit a strong hTERT-specific cytotoxic T lymphocyte (CTL) response [Liao Z L, et al. Cancer Sci. 2012; 103(11): 1920-8]. A vaccine containing hTERT I540 peptide (ILAKFLHWL), hTERT R572Y peptide (YLFFYRKSV), hTERT D988Y peptide (YLQVNSLQTV), survivin Sur1M2 peptide (LMLGEFLKL) and cytomegalovirus control peptide N495 (NLVPMVATV) is vaccinated into myeloma patients transplanted with autologous stem cells, further eliciting strong T cell recovery and sustained reduction of regulatory T cells (Tregs) [Rapoport A P, et al. Blood 2011; 117(3): 788-97]. hTERT peptide vaccines, such as the GV1001 vaccine, have shown promising results in some clinical trials of cancer therapy, but still they cannot induce anti-cancer responses in patients with cutaneous T cell lymphoma [Schlapbach C, et al. J Dermatol Sci. 2011; 62(2): 75-83]. Also, the GV1001 vaccine used in pancreatic cancer patients during chemotherapy fails to improve overall survival of patients [Middleton G, et al. Lancet Oncol. 2014; 15(8): 829-40].

Mucin 1 (MUC1) is mostly type I transmembrane protein with an O-glycosidic bond connected to peptide core. Under normal circumstances, MUC1 is mainly expressed near luminal epithelial cells or glandular surface of many tissues and organs, showing at apical surface of epithelial cells. Due to its abnormal expression in 80-90% tumor tissues, thus MUC1 becomes a potential target for anti-cancer therapy [Pillai K, et al. Am J Clin Oncol. 2015; 38(1): 108-18]. However, MUC1 (amino acid residue 130-154) peptide vaccine tecemotide used for the immunotherapy in the phase III non-small cell lung cancer (NSCLC) patients without resection fails to improve the survival of NSCLC patients in clinical trials [Butts C, et al. Lancet Oncol. 2014; 15(1): 59-68]. Probably a vaccine comprising a single tumor-associated antigen (TAA) such as MUC1 for the immunotherapy in NSCLC patients may be invalid [Xia W, et al. J Thorac Dis. 2014; 6(10): 1513-20].

Ras gene family associated with human tumors includes Hras, Kras and Nras. Among them, Kras greatly impacts on human cancer and is like a “switch” of the body. Under normal circumstances, Kras can regulate cell growth; under abnormal circumstances, Kras causes continuous growth of cells and prevents self-destruction of cells. Currently, some chemotherapeutic drugs targeting Kras have entered the clinical use, but these drugs are prone to drug resistance. A potential way is to treat cancer through vaccination. It is demonstrated that dendritic cell (DC) vaccines containing the entire antigens from PANC cells (with Kras point mutations) can induce a good anti-cancer immune response, but vaccines containing normal cell components may cause immune tolerance and autoimmune response [Tan G, et al. Oncol Rep. 2011; 26(1): 215-21]. DC vaccines pulsed with Kras (12 Val) mutant peptide can promote the expression of mature DC surface molecules and enhance cytotoxic T lymphocyte (CTL) responses, but fails to achieve a strong anti-cancer immune effect. Therefore, the above DC vaccines pulsed with Kras (12 Val) mutant peptide still require to be improved.

Epidermal growth factor receptor (EGFR) is a receptor for epidermal growth factor (EGF). EGFR is expressed on the surface of normal epithelial cells and abnormally expressed in some tumor cells. Over expression of EGFR is related to tumor cell metastasis, invasion and poor prognosis. EGFR-tyrosine kinase inhibitors such as gefitinib and erlotinib used for NSCLC patients with EGFR mutant have proven the significant clinical activity. However, most cancer patients can develop drug resistance. EGFR T790M mutation (the threonine to methionine change at codon 790 of EGFR) is the most prevalent drug resistance mutation. A peptide vaccine containing EGFR T790M mutant is used for the immunotherapy in NSCLC patients, revealing that the immunotherapy of targeting EGFR T790M mutant antigen may be a new option for the treatment of NSCLC patients with EGFR T790M mutation [Ofuji K, et al. Int J Oncol. 2015; 46(2): 497-504]. A DNA vaccine encoding Kras mutant gene can elicit an effective immune response against the tumor with Kras mutation, but is invalid for the tumor with EGFR mutation [Weng T Y, et al. Gene Ther. 2014; 21(10): 888-96]. Also, gefitinib and erlotinib are valid only for the treatment of NSCLC patients with EGFR mutation, but invalid for NSCLC patients with both EGFR mutation and Kras mutation. If simultaneously targeting both EGFR mutation and Kras mutation, the effects of cancer treatment may be multiplied.

In summary, DNA cancer vaccines may integrate into the host cell’ genome and produce the insertional mutation because DNA cancer vaccines require to enter the host cellular nucleus and be transcribed into mRNA, which is transported into the cytoplasm and translated into the corresponding protein. Viral vector-based cancer vaccines may cause serious side effects. Full sequence of tumor-associated antigens (TAAs) can elicit strong anti-cancer immune responses, but may induce immune tolerance and auto-immune responses. A single epitope (or peptide) vaccine may not elicit a strong enough immune response, e.g., the GV1001 vaccine used in patients with cutaneous T cell lymphoma and in pancreatic cancer patients during chemotherapy cannot achieve treatment effects. It is often ineffective to treat NSCLC patients with a single epitope vaccine such as MUC1 peptide vaccine. Multiple epitopes such as hTERT I540/R572Y/D988Y combined vaccine, tetramers constituted by multiple epitopes (e.g., hTERT 540-548, 572Y-580, 865-873 tetramer), and multiple antigenic peptides have better immunotherapeutic effects than that of a single epitope vaccine. A neoantigen cancer vaccine has a good immunotherapeutic effect. Due to the lack of strong immune adjuvants in the above mentioned vaccines, cancer vaccines still require to be improved to achieve the desired immunotherapeutic effects.

### SUMMARY OF THE INVENTION

The object of the present invention is to provide an mRNA cancer vaccine encoding human GM-CSF fused to multiple tandem epitopes.

To achieve the object of the present invention, pVec-GM-CSF-hTes, pVec-GMKE and pVec-hIL-12 are respectively constructed and used as templates for generating the corresponding in vitro transcribed (IVT) mRNAs. The obtained IVT mRNAs are electroporated into the cells for detecting the expression and further mixed together as an mRNA cancer vaccine. This mRNA cancer vaccine contains human GM-CSF used as an immune adjuvant, multiple tandem epitopes constituting as multi-epitope cancer antigens and hIL-12 used to enhance the immunotherapeutic effects.

### BRIEF DESCRIPTION OF THE DRAWINGS

FIG. 1 pVec-GM-CSF-hTes map

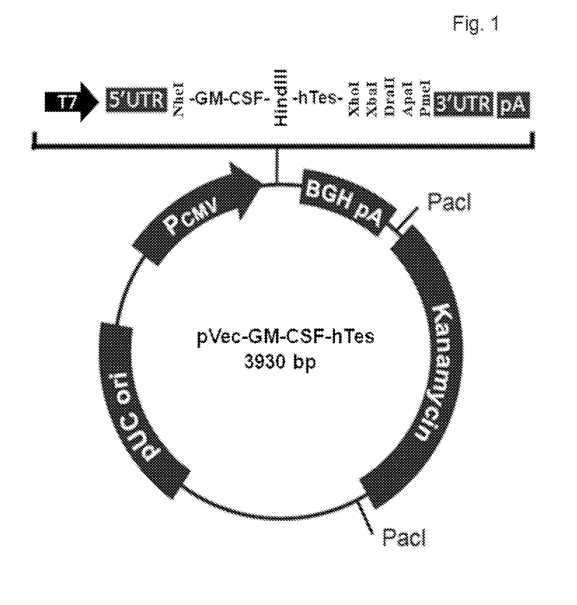

Human GM-CSF (without a stop codon)-linker-three tandem hTERT epitopes (with a stop codon) is subcloned between NheI and XhoI sites of pVec.

Full nucleotide sequence of pVec-GM-CSF-hTes: 3,930 bp

GM-CSF-hTes: bases 801-1,391 bp

FIG. 2 pVec-GMKE map

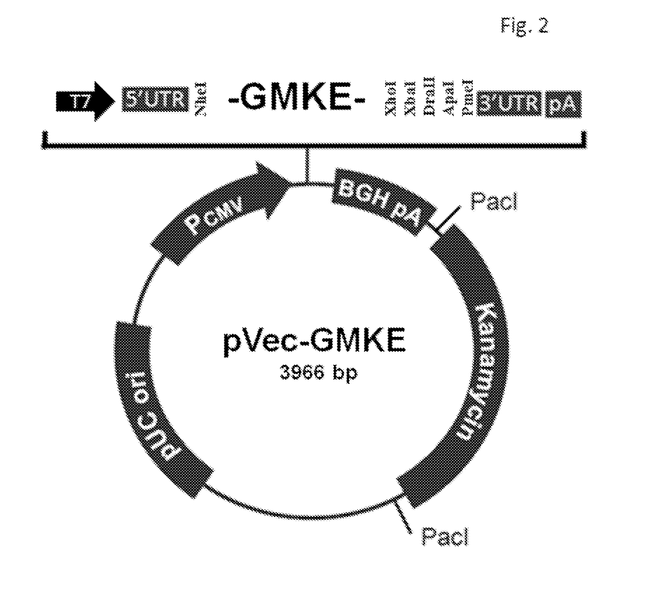

GMKE which stands for human GM-CSF fused to three tandem epitopes respectively from MUC1, Kras and EGFR is subcloned between NheI and XhoI sites of pVec.

Full nucleotide sequence of pVec-GMKE: 3,966 bp

GMKE: bases 801-1,427 bp

FIG. 3 pVec-hIL-12 map

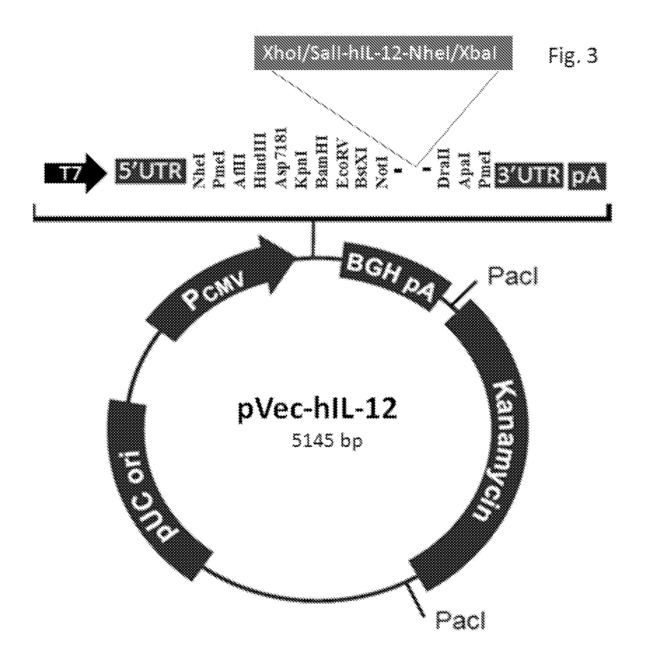

SalI-hIL-12-NheI is subcloned between XhoI and XbaI sites of pVec.

Full nucleotide sequence of pVec-hIL-12: 5,145 bp

### DETAILED DESCRIPTION OF THE INVENTION

The object of the present invention is to provide an mRNA cancer vaccine encoding human granulocyte macrophage colony-stimulating factor (GM-CSF) fused to multiple tandem epitopes, which is obtained using conventional molecular biotechnologies through the following steps.

Taking pCMV-SPORT6-GM-CSF (purchased from Open Biosystems, GM-CSF GenBank accession number: BC108724) as a template, and using the forward primer designed according to Kozak sequence as SEQ ID NO: 1 and the reverse primer designed by deleting human GM-CSF stop codon (tga) and adding a linker (SEQ ID NO: 2) to the 3′ end as SEQ ID NO: 3, the product obtained by polymerase chain reaction (PCR) amplification is subcloned into NheI and HindIII sites of our proprietary pVec, and transformed into top10 chemically competent E. coli cells or DH5 alpha competent cells, obtaining pVec-NheI-GM-CSF (without a stop codon)-linker-HindIII.

pYEX-BX encoding KAP123-flu (purchased from Addgene, plasmid number: 24048) is digested with restriction endonuclease San. Subsequently, the fragment containing vector backbone is isolated by 1% agarose gel electrophoresis, self-ligated with T4 DNA ligase by head to tail connection and transformed into top10 chemically competent E. coli cells or DH5 alpha competent cells, obtaining pYEX-BX vector.

Three epitopes including 1540-548 (SEQ ID NO: 4), 572Y-580 (SEQ ID NO: 5) and 988Y-997 (SEQ ID NO: 6) are selected from hTERT (GenBank accession number: AF015950). Two linkers including an 11 amino acid (aa) linker (SEQ ID NO: 7) and a 2 amino acid linker (GlyGly) are designed and used to tandem connect the above three hTERT epitopes.

The designed amino acid sequence of hTERT (1540-548)-11 aa-hTERT (572Y-580)-2 aa-hTERT (988Y-997) is as SEQ ID NO: 8 and the corresponding nucleotide sequence (with a start codon, atg) is as SEQ ID NO: 9.

To obtain the fragment containing BamHI-hTERT (1540-548)-11 aa-hTERT (572Y-580)-2 aa-hTERT (988Y-997)-SalI, the following designed oligonucleotides are synthesized and ligated.

hTERT F1 nucleotide sequence is as SEQ ID NO: 10.

hTERT F2 nucleotide sequence is as SEQ ID NO: 11.

hTERT F3 nucleotide sequence is as SEQ ID NO: 12.

hTERT R1 nucleotide sequence is as SEQ ID NO: 13.

hTERT R2 nucleotide sequence is as SEQ ID NO: 14.

hTERT R3 nucleotide sequence is as SEQ ID NO: 15.

Two μg of pYEX-BX is digested with BamHI and SalI, dephosphorylated with alkaline phosphatase (calf intestinal, CIP, New England Biolabs, Cat #: M0290S) and purified.

All the above indicated oligonucleotides (0.25 μg/each oligonucleotide), 2.5 μl of 10× reaction buffer, 2 μl T4 polynucleotide kinase (New England Biolabs, Cat #: M0201S) and the appropriate amount of water to a total volume of 25 μl are put into a PCR reaction tube. After mixing, the above reaction tube is incubated for phosphorylation at 37° C. for 1 hour, subsequently denatured at 94° C. for 10 minutes, annealed at room temperature for 30 minutes and then put on ice for 10 minutes.

Four μl of the above annealed reaction products, an equal amount of pYEX-BX vector digested with BamHI and SalI and dephosphorylated, 2 μl of 10×T4 ligation buffer, 1 μl T4 DNA ligase, and the appropriate water to a total volume of 20 μl are put into a PCR reaction tube, incubated at 16° C. overnight and then transformed into top10 chemically competent E. coli cells or DH5 alpha competent cells, obtaining pYEX-BX-BamHI-hTERT (1540-548)-11 aa-hTERT (572Y-580)-2 aa-hTERT (988Y-997)-SalI.

Taking the above constructed vector pYEX-BX-BamHI-hTERT (1540-548)-11 aa-hTERT (572Y-580)-2 aa-hTERT (988Y-997)-SalI as a template, and using the forward primer deleting a start codon (atg) as SEQ ID NO: 16 and the reverse primer adding a stop codon (tga) as SEQ ID NO: 17, the fragment containing hTERT (1540-548)-11 aa-hTERT (572Y-580)-2 aa-hTERT (988Y-997)-stop codon (tga) is obtained by PCR amplification, and then subcloned into HindIII and XhoI sites of pVec-NheI-GM-CSF (without a stop codon)-linker-HindIII, and transformed into top10 chemically competent E. coli cells or DH5 alpha competent cells, obtaining pVec-NheI-GM-CSF (without a stop codon)-linker-HindIII-hTERT (1540-548)-11 aa-hTERT (572Y-580)-2 aa-hTERT (988Y-997)-stop codon (tga)-XhoI, referred to as pVec-GM-CSF-hTes. pVec-GM-CSF-hTes is deposited as PTA-122795 at the American Type Culture Collection (ATCC).

The nucleotide sequence of hTERT (1540-548)-11 aa-hTERT (572Y-580)-2 aa-hTERT (988Y-997) of pVec-GM-CSF-hTes is as SEQ ID NO: 18. The nucleotide sequence of GM-CSF (without a stop codon)-linker-HindIII-hTERT (1540-548)-11 aa-hTERT (572Y-580)-2 aa-hTERT (988Y-997) or GM-CSF-hTes of pVec-GM-CSF-hTes is as SEQ ID NO: 19, the corresponding amino acid sequence is as SEQ ID NO: 20. The full nucleotide sequence of pVec-GM-CSF-hTes has been sequenced by Genewiz Company and is as SEQ ID NO: 21.

The amino acid sequence of MUC1 (aa 130-154) selected from MUC1 (GenBank accession number: J05582) is as SEQ ID NO: 22, and the corresponding nucleotide sequence is as SEQ ID NO: 23.

The amino acid sequence of Kras 12 Val (aa 5-17) selected from Kras (GenBank accession number: M54968) is as SEQ ID NO: 24, and the corresponding nucleotide sequence is as SEQ ID NO: 25.

The amino acid sequence of EGFR T790M (aa 788-798) selected from EGFR (GenBank accession number: GU255993) is as SEQ ID NO: 26, and the corresponding nucleotide sequence is as SEQ ID NO: 27.

The amino acid sequence of the linker used to tandem connect the above mentioned epitopes is as Gly Gly, and the corresponding nucleotide sequence is as gga ggt.

The amino acid sequence of the designed MUC1 (aa 130-154)-2 aa-Kras 12 Val (aa 5-17)-2 aa-EGFR T790M (aa 788-798) is as SEQ ID NO: 28, and the corresponding nucleotide sequence is as SEQ ID NO: 29. In addition, a stop codon (tga) is added to the 3′ end.

The inserter containing MUC1 (aa 130-154)-2 aa-Kras 12 Val (aa 5-17)-2 aa-EGFR T790M (aa 788-798)-stop codon (tga) is gradually subcloned into HindIII and XhoI sites of pVec-NheI-GM-CSF (without a stop codon)-linker-HindIII, transformed into top10 chemically competent E. coli cells or DH5 alpha competent cells.

Taking pVec-NheI-GM-CSF (without a stop codon)-linker-HindIII as a template and using the above mentioned forward primer as SEQ ID NO: 1 as well as the reverse primer as SEQ ID NO: 30, the product obtained by PCR amplification is subcloned into NheI and XhoI sites of pVec-NheI-GM-CSF (without a stop codon)-linker-HindIII, and transformed into top10 chemically competent cells or DH5 alpha competent cells, obtaining pVec-NheI-GM-CSF (without a stop codon)-linker-HindIII-MUC1a-XhoI.

Taking the above obtained pVec-NheI-GM-CSF (without a stop codon)-linker-HindIII-MUC1a-XhoI as a template and using the mentioned forward primer as SEQ ID NO: 1 as well as the reverse primer as SEQ ID NO: 31, the product obtained by PCR amplification is subcloned into NheI and XhoI sites of pVec-NheI-GM-CSF (without a stop codon)-linker-HindIII, and transformed into top10 chemically competent cells or DH5 alpha competent cells, obtaining pVec-NheI-GM-CSF (without a stop codon)-linker-HindIII-MUC1-XhoI.

Taking the above obtained pVec-NheI-GM-CSF (without a stop codon)-linker-HindIII-MUC1-XhoI as a template and using the mentioned forward primer as SEQ ID NO: 1 as well as the reverse primer as SEQ ID NO: 32, the product obtained by PCR amplification is subcloned into NheI and XhoI sites of pVec-NheI-GM-CSF (without a stop codon)-linker-HindIII, and transformed into top10 chemically competent cells or DH5 alpha competent cells, obtaining pVec-NheI-GM-CSF (without a stop codon)-linker-HindIII-MUC1-2 aa-Kras 12 Val-2 aa-XhoI.

Taking the above obtained pVec-NheI-GM-CSF (without a stop codon)-linker-HindIII-MUC1-2 aa-Kras 12 Val-2 aa-XhoI as a template and using the mentioned forward primer as SEQ ID NO: 1 as well as the reverse primer as SEQ ID NO: 33, the product obtained by PCR amplification is subcloned into NheI and XhoI sites of pVec-NheI-GM-CSF (without a stop codon)-linker-HindIII, and transformed into top10 chemically competent cells or DH5 alpha competent cells, obtaining pVec-NheI-GM-CSF (without a stop codon)-linker-HindIII-MUC1 (aa 130-154)-2 aa-Kras 12 Val (aa 5-17)-2 aa-EGFR T790M (aa 788-798)-stop codon (tga)-XhoI, referred to as pVec-GMKE. pVec-GMKE is deposited as PTA-122796 at the American Type Cell Collection (ATCC).

The amino acid sequence of GM-CSF (without a stop codon)-linker-HindIII-MUC1 (aa 130-154)-2 aa-Kras 12 Val (aa 5-17)-2 aa-EGFR T790M (aa 788-798) or GMKE of pVec-GMKE is as SEQ ID NO: 34, and the corresponding nucleotide sequence is as SEQ ID NO: 35. The full nucleotide sequence of pVec-GMKE has been sequenced by Genewiz Company and is as SEQ ID NO: 36.

Human interleukin-12 (hIL-12) gene is obtained by digesting pORF-hIL-12 G2 (InvivoGen) with SalI and NheI, and subcloned into XhoI and XbaI sites of pVec, obtaining pVec-hIL-12. The complete nucleotide sequence of pVec-hIL-12 is as SEQ ID NO: 37.

The above obtained pVec-GM-CSF-hTes, pVec-GMKE and pVec-hIL-12 are amplified, purified with Qiaprep spin miniprep kit (Qiagen, Cat #: 27106), digested with restriction endonuclease SpeI respectively, obtaining the corresponding linearized plasmid DNAs. A small amount of each of the above SpeI cut plasmid DNAs is used to detect whether each of the plasmid DNAs is completely linearized by 1% agarose gel electrophoresis. Each of the obtained linearized plasmid DNAs is purified through the following protocol. The mixture of 100 μl SpeI cut plasmid DNA reaction solution with 500 μl Buffer PB is transferred into a spin column, centrifuging for 30 seconds and discarding the effluent (flow-through). 750 μl Buffer PE is added to the above spin column, centrifuging for 30 seconds, draining the effluent and then centrifuging again for 1 minute. The above spin column is put into a clean micro-centrifuge tube, adding 30 μl of water to the spin column, standing for 1 minute and centrifuging for 1 minute. The purified linearized plasmid DNA is used to determine the DNA concentration and adjust to the concentration of 0.5 to 1 μg/μ1.

Using HiScribe™ T7 High Yield RNA Synthesis Kit (New England Biolabs, Cat #: E2040S) and 3′-0-Me-m⁷G(5′)ppp(5′)G RNA Cap Structure Analog (ARCA, New England Biolabs, Cat #: S1411S), the IVT GM-CSF-hTes mRNA, GMKE mRNA and hIL-12 mRNA are respectively generated through the following steps.

At room temperature, the following reagents are respectively added to a 1.5 ml micro-centrifuge tube.

| | | |
|---|---|---|
| Nuclease-free water | x μl | |
| 10 X reaction buffer | 2 μl | |
| ATP (100 mM) | 2 ul | 10 mM final |
| UTP (100 mM) | 2 μl | 10 mM final |
| CTP (100 mM) | 2 μl | 10 mM final |
| GTP (20 mM) | 2 μl | 2 mM final |
| ARCA (40 mM) | 4 μl | 8 mM final |
| Template DNA (linearized) | x μl | 1 μg |
| T7 RNA polymerase mix | 2 μl | |
| Total reaction volume | 20 μl | |

After mixing well and pulse-spinning, the above reaction tube is incubated at 37° C. for 2 hours. To remove the template DNA, 70 μl nuclease-free water, 10 μl of 10×DNase I buffer and 2 μl DNase I (New England Biolabs, Cat #: M0303S) are added to the above reaction tube, incubating at 37° C. for 15 minutes.

Using RNeasy mini kit (Qiagen, Cat #: 74104), the IVT GM-CSF-hTes mRNA, GMKE mRNA and hIL-12 mRNA are respectively purified through the following steps. 20 to 30 μl of the IVT mRNA diluted with nuclease-free water is taken and transferred into a micro-centrifuge tube (nuclease-free) each time, 350 μl Buffer RLT with 1% β-mercaptoethanol (β-ME) is added to the above tube. After thoroughly mixing with pipette, an equal volume of 70% ethanol is added to the above tube. After mixing, the above mixture is transferred into a spin column for centrifuging and draining the effluent (flow-through). 700 μl Buffer RW1 is added to the above spin column, centrifuging and draining the effluent. 500 μl Buffer RPE is added to the above spin column, centrifuging and draining the effluent, repeating twice. After centrifuging again for 1 minute, the above spin column is transferred into a clean micro-centrifuge tube (nuclease-free) and 30 μl of nuclease-free water is added to the above spin column, standing for 1 minute and then centrifuging for 1 minute. The purified IVT mRNA is used to determine the mRNA concentration using a Nanodrop spectrophotometer and its quality is detected by 1% formaldehyde agarose gel electrophoresis.

Each of the purified IVT GM-CSF-hTes mRNA (5 μg), GMKE mRNA (5 μg) and hIL-12 mRNA (5 μg) is respectively electroporated into 1×10⁶ cells (e.g., mouse cell lines) in a 0.2 cm cuvette at the condition of 350 V, 500 μs. Subsequently the cells electroporated with the IVT mRNA are cultured in a cell growth medium at 5% CO₂, 37° C. for 36 hours and then the above cell supernatants are collected.

The collected supernatants of the cells electroporated with GM-CSF-hTes mRNA and the cells with GMKE mRNA are respectively used to detect human GM-CSF expression with human GM-CSF enzyme-linked immunosorbent assay (ELISA) kit (eBioscience, Cat #: 88-8337-22) through the following steps.

The ELISA plate is coated with 100 μl capture antibody diluted with 1× coating buffer at the ratio of 1:250 each well, sealed and put at 4° C. overnight.

Next day, discarding the excess capture antibody solution and washing the above ELISA plate with wash buffer [1× phosphate-buffered saline (PBS) containing 0.05% Tween-20] 3 times, at least 1 minute each time and patting dry, 200 μl of 1×ELISA/ELISPOT Diluent is added to each well of the above plate, then incubating at room temperature for 1 hour.

The above ELISA plate is washed following the previous indicated method once. 100 μl of 1×ELISA/ELISPOT Diluent diluted standard human GM-CSF or 100 μl of the collected cell supernatant is added to each well, then sealing and incubating at room temperature for 2 hours.

The above plate is washed according to the above indicated method 3 to 5 times. 100 μl of 1×ELISA/ELISPOT Diluent diluted detection antibody is added to each well, then sealing and incubating at room temperature for 1 hour.

The above plate is washed according to the previous mentioned method 3 to 5 times. 100 μl of 1×ELISA/ELISPOT Diluent diluted Avidin-horseradish peroxidase (HRP) is added to each well, then sealing and incubating at room temperature for 30 minutes.

The plate is washed according to the above indicated method 5 to 7 times. 100 μl of 1× tetramethylbenzidine (TMB) solution is added to each well, incubating at room temperature for 15 minutes.

Then 50 μl of 2 M H₂SO₄ stop solution is added to each well of the above ELISA plate. Further, the concentration of human GM-CSF expressed in the cell supernatant is determined by measuring optical density (OD) value at 450 nm using a micro-plate reader.

The experimental results show that both the cells electroporated with GM-CSF-hTes mRNA and the cells with GMKE mRNA can express human GM-CSF.

The collected supernatants from the cells electroporated with the IVT hIL-12 mRNA are used to detect human IL-12 expression with human IL-12 ELISA kit (eBioscience, Cat #: 88-7126-88) by the previous mentioned protocol.

The ELISA plate is coated with 100 μl capture antibody diluted with 1× coating buffer at the ratio of 1:250 for each well, sealed and incubated at 4° C. overnight.

After discarding the coating solution containing capture antibody, rinsing with wash buffer (1×PBS containing 0.05% Tween-20) 3 times, at least 1 minute each time, and patting dry, 200 μl of 1×ELISA/ELISPOT Diluent is added to each well of the above plate, then incubating at room temperature for 1 hour.

According to the previous mentioned method, the above plate is washed. 100 μl of 1×ELISA/ELISPOT Diluent diluted standard human IL-12 or 100 μl of the collected supernatant is added to each well, then sealing and incubating at room temperature for 2 hours.

The plate is washed according to the previous method 3 to 5 times. 100 μl of 1×ELISA/ELISPOT Diluent diluted detection antibody is added to each well, then sealing and incubating at room temperature for 1 hour.

The plate is washed according to the above method 3 to 5 times. 100 μl of 1×ELISA/ELISPOT Diluent diluted Avidin-HRP is added to each well of the above plate, sealing and incubating at room temperature for 30 minutes.

The plate is washed according to the above method 5 to 7 times, 100 μl of 1×TMB solution is added to each well, then incubating at room temperature for 15 minutes.

Then 50 μl of 2 M H₂SO₄ stop solution is added to each well of the above plate. Further, the concentration of human IL-12 expressed in the cell supernatant is determined by measuring OD value at 450 nm using a micro-plate reader.

The experimental results show that the cells electroporated with the IVT hIL-12 mRNA can express human IL-12.

The percentage identity between a query sequence and a subject is obtained using basic local alignment search tool (BLAST).

The mRNA components of an mRNA cancer vaccine encoding human GM-CSF fused to multiple tandem epitopes include GM-CSF-hTes mRNA, GMKE mRNA and hIL-12 mRNA. This mRNA cancer vaccine contains human GM-CSF used as an immune adjuvant, multiple tandem epitopes constituting as multi-epitope cancer antigens and hIL-12 used to enhance the immunotherapeutic effects. Therefore, this mRNA cancer vaccine would be a very effective vaccine for cancer immunotherapy, especially targeting non-small cell lung cancer (NSCLC) patients.

1. The pVec-GM-CSF-hTes, wherein the complete nucleotide sequence of pVec-GM-CSF-hTes is at least 65% identical to SEQ ID NO: 21.

2. The pVec-GM-CSF-hTes of claim 1, wherein the nucleotide sequence of the reverse primer used for PCR amplification and obtaining human GM-CSF (without a stop codon) is at least 69% identical to SEQ ID NO: 3.

3. The pVec-GM-CSF-hTes of claim 1, wherein the nucleotide sequence of the forward primer used for PCR amplification and obtaining hTERT (1540-548)-11 aa-hTERT (572Y-580)-2 aa-hTERT (988Y-997) is at least 69% identical to SEQ ID NO: 16.

4. The pVec-GM-CSF-hTes of claim 1, wherein the nucleotide sequence of the reverse primer used for PCR amplification and obtaining hTERT (1540-548)-11 aa-hTERT (572Y-580)-2 aa-hTERT (988Y-997) is at least 68% identical to SEQ ID NO: 17.

5. The pVec-GM-CSF-hTes of claim 1, wherein the amino acid sequence of hTERT (1540-548)-11 aa-hTERT (572Y-580)-2 aa-hTERT (988Y-997) is at least 39% identical to SEQ ID NO: 8.

6. The pVec-GM-CSF-hTes of claim 1, wherein the nucleotide sequence of hTERT (1540-548)-11 aa-hTERT (572Y-580)-2 aa-hTERT (988Y-997) is at least 32% identical to SEQ ID NO: 18.

7. The pVec-GM-CSF-hTes of claim 1, wherein the amino acid sequence of GM-CSF-hTes is at least 78% identical to SEQ ID NO: 20.

8. The pVec-GM-CSF-hTes of claim 1, wherein the nucleotide sequence of GM-CSF-hTes is at least 76% identical to SEQ ID NO: 19.

9. The pVec-GMKE, wherein the complete nucleotide sequence of pVec-GMKE is at least 64% identical to SEQ ID NO: 36.

10. The pVec-GMKE of claim 9, wherein the amino acid sequence of MUC1 (aa 130-154)-2 aa-Kras 12 Val (aa 5-17)-2 aa-EGFR T790M (aa 788-798) is at least 52% identical to SEQ ID NO: 28.

11. The pVec-GMKE of claim 9, wherein the nucleotide sequence of MUC1 (aa 130-154)-2 aa-Kras 12 Val (aa 5-17)-2 aa-EGFR T790M (aa 788-798) is at least 49% identical to SEQ ID NO: 29.

12. The pVec-GMKE of claim 9, wherein the amino acid sequence of GMKE is at least 72% identical to SEQ ID NO: 34.

13. The pVec-GMKE of claim 9, wherein the nucleotide sequence of GMKE is at least 71% identical to SEQ ID NO: 35.


SEQUENCE LISTING (37 sequences)

SEQ ID NO: 1 - DNA, 59 bp, Homo sapiens
  Primer (1)..(59)
acgtgctagcgccgccaccatggggctgcagagcctgctgctcttgggcactgtggcct

SEQ ID NO: 2 - protein, 9 aa, Artificial Sequence
  Synthesized
GGGGSGGGG

SEQ ID NO: 3 - DNA, 75 bp, Homo sapiens
  Primer (1)..(75)
acgtaagcttcccgcctccaccactacctcccccgccctcctggactggctcccagcagtcaaaggggatgacaa

SEQ ID NO: 4 - protein, 9 aa, Homo sapiens
ILAKFLHWL

SEQ ID NO: 5 - protein, 9 aa, Homo sapiens
YLFFYRKSV

SEQ ID NO: 6 - protein, 10 aa, Homo sapiens
YLQVNSLQTV

SEQ ID NO: 7 - protein, 11 aa, Artificial Sequence
  Synthesized
GGGGSGGGGSG

SEQ ID NO: 8 - protein, 41 aa, Artificial Sequence
  Synthesized
ILAKFLHWLGGGGSGGGGSGYLFFYRKSVGGYLQVNSLQTV

SEQ ID NO: 9 - DNA, 126 bp, Artificial Sequence
  Synthesized
atgatcctggccaagttcctgcactggctgggtggcggagggtctggtggcggagggtctggttatctctttttctaccggaagagtgtcggtggctatttgcaggtgaacagcctccagacggtg

SEQ ID NO: 10 - DNA, 41 bp, Artificial Sequence
  Synthesized
gatccatgatcctggccaagttcctgcactggctgggtggc

SEQ ID NO: 11 - DNA, 45 bp, Artificial Sequence
  Synthesized
ggagggtctggtggcggagggtctggttatctctttttctaccgg

SEQ ID NO: 12 - DNA, 45 bp, Artificial Sequence
  Synthesized
aagagtgtcggtggctatttgcaggtgaacagcctccagacggtg

SEQ ID NO: 13 - DNA, 41 bp, Artificial Sequence
  Synthesized
tcgaccaccgtctggaggctgttcacctgcaaatagccacc

SEQ ID NO: 14 - DNA, 45 bp, Artificial Sequence
  Synthesized
gacactcttccggtagaaaaagagataaccagaccctccgccacc

SEQ ID NO: 15 - DNA, 46 bp, Artificial Sequence
  Synthesized
agaccctccgccacccagccagtgcaggaacttggccaggatcatg

SEQ ID NO: 16 - DNA, 40 bp, Artificial Sequence
  Synthesized
  Primer (1)..(40)
acgtaagcttatcctggccaagttcctgcactggctgggt

SEQ ID NO: 17 - DNA, 46 bp, Artificial Sequence
  Synthesized
  Primer (1)..(46)
acgtctcgagtcacaccgtctggaggctgttcacctgcaaatagcc

SEQ ID NO: 18 - DNA, 123 bp, Artificial Sequence
  Synthesized
atcctggccaagttcctgcactggctgggtggcggagggtctggtggcggagggtctggttatctctttttctaccggaagagtgtcggtggctatttgcaggtgaacagcctccagacggtg

SEQ ID NO: 19 - DNA, 591 bp, Artificial Sequence
  Synthesized
atggggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcgggggaggtagtggtggaggcgggaagcttatcctggccaagttcctgcactggctgggtggcggagggtctggtggcggagggtctggttatctctttttctaccggaagagtgtcggtggctatttgcaggtgaacagcctccagacggtgtga

SEQ ID NO: 20 - protein, 196 aa, Artificial Sequence
  Synthesized
MGLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGKLILAKFLHWLGGGGSGGGGSGYLFFYRKSVGGYLQVNSLQTV

SEQ ID NO: 21 - DNA, 3930 bp, Artificial Sequence
  Synthesized
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagattggaccctcgtacagaagctaatacgactcactatagggaaataagagagaaaagaagagtaagaagaaatataagagccaccgctagcgccgccaccatggggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcgggggaggtagtggtggaggcgggaagcttatcctggccaagttcctgcactggctgggtggcggagggtctggtggcggagggtctggttatctctttttctaccggaagagtgtcggtggctatttgcaggtgaacagcctccagacggtgtgactcgagtctagagggcccgtttaaacaccggtagctcgcttgtccaatttctattaaaggttcctttgttccctaagtccaactactaaactgggggatattatgaagggccttgagcatctggattctgcctaataaaaaacatttattttcattgcatcgatagctcgctttcttgctgtccaatttctattaaaggttcctttgttccctaagtccaactactaaactgggggatattatgaagggccttgagcatctggattctgcctaataaaaaacatttattttcattgcccgcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaattattactagtccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctttaattaaactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgcttaattaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 22 - protein, 25 aa, Homo sapiens
STAPPAHGVTSAPDTRPAPGSTAPP

SEQ ID NO: 23 - DNA, 75 bp, Homo sapiens
tccaccgcccccccagcccacggtgtcacctcggccccggacaccaggccggccccgggctccaccgccccccca

SEQ ID NO: 24 - protein, 13 aa, Homo sapiens
KLVVVGAVGVGKS

SEQ ID NO: 25 - DNA, 39 bp, Homo sapiens
aagctggtggtggtgggcgccgtgggtgtgggcaagagt

SEQ ID NO: 26 - protein, 11 aa, Homo sapiens
LIMQLMPFGCL

SEQ ID NO: 27 - DNA, 33 bp, Homo sapiens
ctcatcatgcagctcatgcccttcggctgcctc

SEQ ID NO: 28 - protein, 53 aa, Artificial Sequence
  Synthesized
STAPPAHGVTSAPDTRPAPGSTAPPGGKLVVVGAVGVGKSGGLIMQLMPFGCL

SEQ ID NO: 29 - DNA, 159 bp, Artificial Sequence
  Synthesized
tccaccgcccccccagcccacggtgtcacctcggccccggacaccaggccggccccgggctccaccgcccccccaggaggtaagctggtggtggtgggcgccgtgggtgtgggcaagagtggaggtctcatcatgcagctcatgcccttcggctgcctc

SEQ ID NO: 30 - DNA, 90 bp, Artificial Sequence
  Synthesized
  Primer (1)..(90)
acgtctcgagggtgtccggggccgaggtgacaccgtgggctgggggggcggtggaaagcttcccgcctccaccactacctcccccgccct

SEQ ID NO: 31 - DNA, 79 bp, Artificial Sequence
  Synthesized
  Primer (1)..(79)
acgtctcgagtcaacctcctgggggggcggtggagcccggggccggcctggtgtccggggccgaggtgacaccgtgggc

SEQ ID NO: 32 - DNA, 85 bp, Artificial Sequence
  Synthesized
  Primer (1)..(85)
acgtctcgagacctccactcttgcccacacccacggcgcccaccaccaccagcttacctcctgggggggcggtggagcccggggc

SEQ ID NO: 33 - DNA, 76 bp, Artificial Sequence
  Synthesized
  Primer (1)..(76)
acgtctcgagtcagaggcagccgaagggcatgagctgcatgatgagacctccactcttgcccacacccacggcgcc

SEQ ID NO: 34 - protein, 208 aa, Artificial Sequence
  Synthesized
MGLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQEGGGGSGGGGKLSTAPPAHGVTSAPDTRPAPGSTAPPGGKLVVVGAVGVGKSGGLIMQLMPFGCL

SEQ ID NO: 35 - DNA, 627 bp, Artificial Sequence
  Synthesized
atggggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcgggggaggtagtggtggaggcgggaagctttccaccgcccccccagcccacggtgtcacctcggccccggacaccaggccggccccgggctccaccgcccccccaggaggtaagctggtggtggtgggcgccgtgggtgtgggcaagagtggaggtctcatcatgcagctcatgcccttcggctgcctctga

SEQ ID NO: 36 - DNA, 3966 bp, Artificial Sequence
  Synthesized
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagattggaccctcgtacagaagctaatacgactcactatagggaaataagagagaaaagaagagtaagaagaaatataagagccaccgctagcgccgccaccatggggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagggcgggggaggtagtggtggaggcgggaagctttccaccgcccccccagcccacggtgtcacctcggccccggacaccaggccggccccgggctccaccgcccccccaggaggtaagctggtggtggtgggcgccgtgggtgtgggcaagagtggaggtctcatcatgcagctcatgcccttcggctgcctctgactcgagtctagagggcccgtttaaacaccggtagctcgcttgtccaatttctattaaaggttcctttgttccctaagtccaactactaaactgggggatattatgaagggccttgagcatctggattctgcctaataaaaaacatttattttcattgcatcgatagctcgctttcttgctgtccaatttctattaaaggttcctttgttccctaagtccaactactaaactgggggatattatgaagggccttgagcatctggattctgcctaataaaaaacatttattttcattgcccgcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaattattactagtccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctttaattaaactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgcttaattaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 37 - DNA, 5145 bp, Artificial Sequence
  Synthesized
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagattggaccctcgtacagaagctaatacgactcactatagggaaataagagagaaaagaagagtaagaagaaatataagagccaccgctagcgtttaaacttaagcttggtaccgagctcggatcctgcagatatccagcacagtggcggccgctcgactactaaccttcttctctttcctacagctgagatcaccggcgaaggagggccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggaggggctagagggcccgtttaaacaccggtagctcgcttgtccaatttctattaaaggttcctttgttccctaagtccaactactaaactgggggatattatgaagggccttgagcatctggattctgcctaataaaaaacatttattttcattgcatcgatagctcgctttcttgctgtccaatttctattaaaggttcctttgttccctaagtccaactactaaactgggggatattatgaagggccttgagcatctggattctgcctaataaaaaacatttattttcattgcccgcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaattattactagtccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctttaattaaactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgcttaattaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt